(19) United States
(12) Patent Application Publication
(10) Pub. No.: US 2026/0034150 A1
(43) Pub. Date: Feb. 5, 2026

(54) CANNABIDIOL FOR USE IN TREATING OR PREVENTING RECURRENT PERICARDITIS

(71) Applicant: CARDIOL THERAPEUTICS INC., Oakville (CA)

(72) Inventors: Andrew Warwick HAMER, New York, NY (US); James Rhys BOLTON, Sheffield (GB)

(73) Assignee: CARDIOL THERAPEUTICS INC., Oakville, ON (CA)

(21) Appl. No.: 19/100,081

(22) PCT Filed: Aug. 21, 2023
(86) PCT No.: PCT/CA2023/051106

§ 371 Date: Jan. 30, 2025

Related U.S. Application Data
(60) Provisional application No. 63/399,881, filed on Aug. 22, 2022.
(60) Provisional application No. 63/422,226, filed on Nov. 3, 2022.

(51) Int. Cl. A61K 31/00; A61P 9/00; A61P 29/00

(52) U.S. Cl. CPC A61K 31/658; A61P 9/00; A61P 29/00

(57) ABSTRACT

Cannabidiol, or a pharmaceutically acceptable prodrug, derivative, salt, or solvate thereof, for use in treating or preventing recurrent pericarditis is disclosed. The effectiveness of cannabidiol in attenuating the levels of interleukin-1β (IL-1β) and IL-6, and the transcription of pro-IL-1β and NLRP3 mRNA in in vitro models are disclosed. Also disclosed are experiments showing cannabidiol to be effective in preventing an increase in the pericardial space (which is an indicator of pericardial effusion) and the pericardial thickness in an in vivo mouse model of pericarditis.

### CROSS-REFERENCE TO RELATED APPLICATIONS

This application claims priority from U.S. provisional applications 63/399,881 and 63/422,226 filed Aug. 22, 2022 and Nov. 3, 2022, respectively, the entire contents of which are incorporated herein by reference.

### FIELD

The present invention relates generally to therapeutics and methods for treating or preventing cardiovascular diseases, disorders, or conditions and, more particularly, to therapeutics and methods for the treatment or prevention of pericarditis.

### BACKGROUND TO THE INVENTION

Pericarditis results from an intense inflammatory reaction of the pericardium. This reaction involves the activation of a macromolecular intracellular complex called the NLRP3 (NACHT, leucine-rich repeat, and pyrin domain-containing protein 3) inflammasome. The NLRP3 inflammasome, when it senses injury and stress, triggers an inflammatory process that involves the release of proinflammatory cytokines, such as interleukin-1β, that act as downstream mediators.

Pericarditis can be categorized into acute, incessant, recurrent and chronic pericarditis. Acute pericarditis is a clinical syndrome characterized by a single incident that resolves on its own or following treatment. The treatments mostly consist of nonsteroidal anti-inflammatory drugs, aspirin, steroids, colchicine, and anakinra (4, 7, 8, 9). Acute pericarditis was reported in 27.7 cases per 100,000 person-years in an Italian urban area, being responsible for 0.1% of all hospital admissions and 5% of emergency room admissions for chest pain (2). Recurrent pericarditis (RP) is diagnosed when an index acute episode is followed by a symptom-free period of at least 4-6 weeks, followed by a subsequent episode. RP has been reported to occur in 15-30% of peri3carditis patients some of whom go on to experience multiple recurrences. In some cases, recurrences are due to inadequate treatment of the original episode, but, in other cases, they are due to inadequate response to current treatments.

The pathogenesis of pericarditis is not completely understood (3); however, it has been hypothesized that acute pericarditis represents a stereotypical response to an acute injury of the mesothelial cells of the pericardium (4). It is believed to be triggered by an “irritant” such as a virus or cellular debris following a viral infection (5,6). It can also be triggered by cardiac procedures such as cardiac surgery (especially coronary artery bypass grafting), pacemaker insertion, radiofrequency ablation, transcatheter aortic valve implantation, and, rarely, percutaneous coronary intervention (7).

According to Klein et al. (12), the entire contents of which are incorporated herein by reference, there is a high unmet need for FDA-approved, safe, accessible treatments that resolve recurrent events and reduce recurrence risk without posing excessive treatment burden on patients. Since the publication of Klein et al., the FDA approved rilonacept (Arcalyst™), an interleukin-1 (IL-1) receptor blocker, as an orphan drug for the treatment of recurrent pericarditis (RP) and reduction in risk of recurrence in adults and pediatric patients 12 years and older (11).

Cannabidiol is a small molecule, lipophilic, non-psychoactive compound found naturally in the plant Cannabis sativa. Interest in this compound has increased over recent years due to its good safety profile and few side effects. Several in vitro and in vivo studies have shown that it possesses anti-inflammatory properties and its use in treating a variety of heart diseases has been suggested in the literature. However, the clinical efficacy of cannabidiol in treating pericarditis is still unknown.

There is an ongoing need for alternative therapeutics and methods of treating recurrent pericarditis. The present invention is intended to meet this need.

### SUMMARY OF THE INVENTION

The inventors have found, through experiments described below, that cannabidiol attenuated an increase in interleukin-1β (IL-1β) in an in vitro model of NLRP3 inflammasome activation. Through another experiment, the inventors found that the anti-inflammatory effect of cannabidiol extends beyond inhibiting the NLRP3 inflammasome pathway as cannabidiol was found to reduce the level of interleukin 6 (IL-6) which is secreted independently of this pathway. Cannabidiol was also found in still other experiments to attenuate an increase in the transcription of pro-IL-1β mRNA and NLRP3 mRNA which are constitutive parts the NLRP3 inflammasome. Furthermore, following in vivo experiments involving a mouse model of pericarditis, the inventors found cannabidiol to be useful in reducing pericardial effusion and pericardial thickness, which are two characteristics of pericarditis.

Accordingly, a first aspect of the invention provides the use of a pharmaceutically active agent chosen from the group comprising cannabidiol and pharmaceutically acceptable prodrugs, derivatives, salts, and solvates thereof, in treating or preventing recurrent pericarditis.

A second aspect of the invention provides the use of a pharmaceutically active agent chosen from the group comprising cannabidiol and pharmaceutically acceptable prodrugs, derivatives, salts, and solvates thereof, in the preparation of a medicament for the treatment or prevention of recurrent pericarditis.

According to a third aspect, the invention provides a method of treating or preventing recurrent pericarditis in a subject in need thereof, comprising (optionally) identifying a subject suffering from or at risk of suffering from recurrent pericarditis, and administering an effective amount of a pharmaceutically active agent chosen from the group comprising cannabidiol and pharmaceutically acceptable prodrugs, derivatives, salts, or solvates thereof, to the subject.

A fourth aspect of the invention provides a pharmaceutically active agent chosen from the group comprising cannabidiol and pharmaceutically acceptable prodrugs, derivatives, salts, and solvates thereof, for use in treating or preventing recurrent pericarditis.

According to a fifth aspect, the invention provides a composition comprising an effective amount of a pharmaceutically active agent chosen from the group comprising cannabidiol and pharmaceutically acceptable prodrugs, derivatives, salts, and solvates thereof, and an effective amount of a delivery vehicle, wherein the composition is for use in treating or preventing recurrent pericarditis.

In some embodiments, the pharmaceutically active agent is synthetic. In the same or other embodiments, the pharmaceutically active agent is cannabidiol.

Some embodiments of the present uses and methods entail using the pharmaceutically active agent to prevent recurrent pericarditis. In other embodiments, the pharmaceutically active agent is used to treat recurrent pericarditis.

The pharmaceutically active agent (e.g. cannabidiol) can be used to attenuate an increase in pericardial effusion and/or pericardial thickness in a subject (e.g. a subject suffering from or at risk of suffering from recurrent pericarditis), for example, by at least about 50, 55, 60, 65, 70, 75, 80, 85, 90, or 95, or up to about 100% as compared to the amount of pericardial effusion and/or pericardial thickness that would be present in the absence of administration of the pharmaceutically active agent.

Furthermore, the pharmaceutically active agent (e.g. cannabidiol) can also be used to attenuate the inflammatory response. For example, it can be used to reduce the transcription of pro-IL-1β mRNA and/or NLRP3 mRNA, which are constitutive parts of the NLRP3 inflammasome. It can also be used to decrease the release of at least one of IL-1β and/or IL-6, in a subject, e.g., by at least about 50, 55, 60, 65, 70, 75, 80, 85, 90, or 95% as compared to the release of IL-1β and/or IL-6 that would occur in the absence of administration of the pharmaceutically active agent.

### BRIEF DESCRIPTION OF THE FIGURES

The invention may be better understood with reference to the following detailed description and figures, wherein,

FIG. 1A is a bar graph showing the effect of cannabidiol at a dose of 10 μM on the level of IL-1β in an in vitro model of NLRP3 inflammasome activation;

FIG. 1B is a bar graph showing the effect of cannabidiol at different doses (10, 1, 0.1, and 0.01 μM) on the level of IL-1β in the same in vitro model of NLRP3 inflammasome activation;

FIG. 1C is a bar graph showing the effect of cannabidiol at a dose of 10 μM on the level of IL-6 in another in vitro model;

FIG. 1D is a bar graph showing the effect of cannabidiol at a dose of 10 μM on the level of transcription of pro-IL-1β mRNA in the same other in vitro model;

FIG. 1E is a bar graph showing the effect of cannabidiol at a dose of 10 μM on the level of transcription of NLRP3 mRNA in the same other in vitro model;

FIG. 2 is a bar graph showing the effect of cannabidiol at two doses (10 mg/kg and 1 mg/kg) on the pericardial space (an indicator of pericardial effusion) in an in vivo model of pericarditis; and

FIG. 3 is a bar graph showing the effect of cannabidiol (10 mg/kg) on the pericardial thickness in the same in vivo model of pericarditis.

### DETAILED DESCRIPTION

### Definitions

For the sake of clarity and to avoid ambiguity, certain terms are defined herein as follows.

As used herein the term “pharmaceutically active agent” means a drug or agent which can be employed as disclosed herein and is intended to be used in a subject to treat or prevent diseases, ailments, physical damage, or pathological symptoms; or to influence the state, the condition or the functions of the body. Drugs in use can be found in reference works such as, for example, the Rote Liste or the Merck Index. Examples which may be mentioned, include, for example, cannabidiol.

When a composition of matter is described as having a “purity of at least X %” this means that one or more impurities may be present in an amount up to 100-X % by weight, based on the total weight of the composition of matter. The purity of an ingredient can be determined by high performance liquid chromatography (HPLC) or other suitable means.

The term “subject” means members of the animal kingdom including humans and other mammals. In some embodiments, the subject is a human.

When used herein, the terms “treat,” “treatment,” and the like expression mean stopping, delaying the progression of a condition, disorder, or disease, or reducing the symptoms or severity of the symptoms thereof. The terms “prevent,” “prevention,” and the like expression mean preventing, delaying, or reducing the incidence and/or risk of the onset of a condition, disorder, or disease. The terms are intended to encompass “improving quality of life,” “extending the life,” and “improving clinical outcomes” of a subject suffering from, or at risk of suffering from, the condition, disorder, or disease, and do not necessarily mean “curing” the condition, disorder, or disease, though such is not precluded.

“Pharmaceutically acceptable excipient” when used herein means any substance which can be formulated with or that is present alongside a pharmaceutically active agent to achieve a desired function or functions and that is not biologically or otherwise undesirable when administered to a subject (e.g. human). For example, the excipient should be non-toxic when administered and compatible with the other ingredients in the formulation. The person skilled in the art will appreciate what compounds or ingredients would qualify as a pharmaceutically acceptable excipient given the teachings of the present specification and information in the public domain.

The phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “a” or “an” means “one or more.” As such, the terms “a” (or “an”), “one or more” and “at least one” can be used interchangeably herein. It should also be noted that the term “or” is employed in the sense of “and/or” unless the context clearly dictates otherwise. For an avoidance of doubt, references herein to the use of cannabidiol to treat or prevent recurrent pericarditis are references to the use of cannabidiol to treat and/or prevent recurrent pericarditis.

The term “comprising” means “including without limitation.” Thus, for example, a composition comprising a list of compounds, ingredients, or elements, may include additional compounds, ingredients, or elements not expressly recited. It is also to be noted that the terms “comprising,” “including,” “containing,” and “having” can be used interchangeably.

The term “consisting of” means including the listed integers and no additional integers except that in the case of a compound or ingredient, other compounds or ingredients that may be present as natural or commercial impurities or additives are not excluded. Natural and commercial impurities and additives will be apparent to the person of ordinary skill in the art. For example, synthetic cannabidiol may contain up to about 0.5% w/w of impurities such as residual solvents and by-products of the manufacturing process.

The term “consisting essentially of” means “including the listed integers (e.g., compounds, steps, ingredients, etc.) and any additional integers that do not materially affect the basic and novel properties of the invention. The basic and novel properties of the invention is the beneficial effect of cannabidiol, pharmaceutically acceptable prodrugs, derivatives, salts, and solvates thereof in treating and/or preventing recurrent pericarditis.

The terms “% w/w,” “% wt.,” “w/w %,” “wt. %” and variations thereof, mean the amount of a substance in terms of the weight of that substance divided by the total weight of the formulation containing that substance, and multiplied by 100.

The terms “% w/v,” “w/v %” and variations thereof, mean the mass of a solute in grams divided by the volume of the solution in which the solute is dissolved in mL, and multiplied by 100. For example, a formulation containing 10 grams of a solvent per 25 mL of the solution that contains the solvent, would have 40 w/v % of the solvent calculated as follows: (10÷25)×100=40 w/v %.

The term “about” refers to variations in an expressed numerical quantity that can occur, for example, through measuring and liquid handling procedures used for making pharmaceutical formulations, differences in the manufacture, source, or purity of the ingredients used to make the formulations, and/or differences due to different equilibrium conditions or different reaction levels of ingredients in a formulation resulting from an initial mixture. For the sake of clarity, the term “about” includes variations in the expressed value up to ±5% or up to ±10%. Whether or not a value is modified by the term “about,” the claims include equivalents to the values.

When used herein, the term “effective amount” means an amount that would bring about the desired effect, based on the purpose and function of the ingredient in the context of the invention disclosed herein. Implicit in “desired effect” is that toxicity does not arise. For example, an effective amount of a pharmaceutically active agent is that amount which would be effective to provide a therapeutic effect while avoiding toxicity, such as may occur via long-term chronic administration. An effective amount of a solvent is that amount which, alone or together with other ingredients, would be effective to solubilize the other or remaining ingredients of the formulation. What constitutes an effective amount will be determinable by the person of ordinary skill in the art by routine experimentation, having regard to the teachings herein and/or information in the public domain.

When used herein, the expression “free of Y” means that “Y” is not deliberately added but may be present as an impurity or due to other factors. For the sake of clarity, a formulation that is “free of Y” will not contain Y or contain only up to 0.01, 0.02, 0.03, 0.04, 0.05, 0.06, 0.07, 0.08, 0.09, 0.1, 0.2, 0.3, 0.4, or 0.5% w/v of Y, based on the total formulation.

The values recited herein are intended to include all values that meet the stated parameters including those not expressly recited. For example, a value of less than 1.0 is intended to include less than 0.99, less than 0.98, less than 0.97, less than 0.90, less than 0.84, less than 0.56, less than 0.01, etc. Thus, all ranges disclosed herein are to be understood to encompass any and all subranges subsumed therein. For example, a stated range of “1 to 10” should be considered to include any and all subranges between (and inclusive of) the minimum value of 1 and the maximum value of 10, e.g., 1 to 6.3, 5.5 to 10, 2.7 to 6.1, etc.

The present specification contemplates the possibility of omitting any ingredients even if they are not expressly named as included or excluded in the specification.

### Cannabidiol

As used herein, the terms “cannabidiol” and “CBD” refer to the compound, 2-[(1R,6R)-3-methyl-6-prop-1-en-2-ylcyclohex-2-en-1-yl]-5-pentylbenzene-1,3-diol, having the below chemical structure:

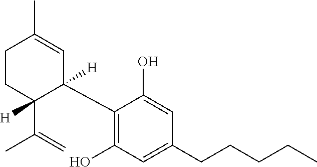 [SMILES: C=C(C)[C@@H]1CCC(C)=C[C@H]1c1c(O)cc(CCCCC)cc1O]

Cannabidiol can be of natural or synthetic origin. Cannabidiol can be derived from a variety of cannabis plants, including hemp, and purified using conventional means. The synthesis of cannabidiol is described, for example, in Petilka et al., Helv. Chim. Acta, 52:1102 (1969) and in Mechoulam et al., J. Am. Chem. Soc., 87:3273 (1965), and U.S. Pat. Nos. 10,059,683 and 10,844,035.

Pharmaceutically acceptable prodrugs, derivatives, salts, and solvates of cannabidiol as are known in the art can also be used in addition to or instead of cannabidiol to treat and/or prevent recurrent pericarditis.

Prodrugs of cannabidiol can include, e.g., a compound that undergoes a chemical conversion, through a metabolic process or otherwise within the body of the mammal receiving the compound, into its active form that has a medical effect. Example cannabidiol prodrugs of the invention include, but are not limited to, cannabidiol esters prepared by functionalizing one or more of the hydroxyl groups that are present within the molecular structure of cannabidiol. Example cannabidiol prodrugs are described in U.S. Pat. No. 8,293,786.

Salt forms of cannabidiol can include any cannabidiol salt that is suitable for administration to a mammal and includes those prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids.

Preferably, the cannabidiol has a purity of at least 99.5%, 99.6%, 99.7%, 99.8% or 99.9%. Impurities that may be present in synthetic cannabidiol include residual solvents (e.g., methanol, n-heptane, dichloromethane, and triethylamine) and/or by-products or residues of manufacture, e.g., olivetol, monobromo-cannabidiol, and tetrahydrocannabinol. Botanically sourced cannabidiol may contain small amounts of other cannabinoids (e.g., cannabidivarin (CBDV) and butyl analog of cannabidiol (cannabidibutol)), terpenes, and solvents or ingredients used in the purification process as well as pesticide residues.

Tetrahydrocannabinol (also referred to as THC, delta-9-tetrahydrocannabinol, and delta-9-THC) is a compound having the structure shown below.

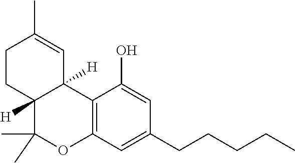 [SMILES: CCCCCc1cc(O)c2c(c1)OC(C)(C)[C@@H]1CCC(C)=C[C@@H]21]

In some embodiments, compositions according to the present invention are free of THC, meaning that THC is either not present or is present in an amount less than 0.5% w/w, preferably less than 0.4% w/w, even more preferably less than 0.3% w/w, and still more preferably less than 0.2% w/w based on the weight of the formulation. To avoid psychotropic or intoxicating effects, it would be desirable to reduce the level of THC to as low a level as possible, e.g., less than 10, 9, 8, 7, 6, 5, 4, 3, 2, or 1 ppm based on the total composition.

The compositions can be in any dosage form known in the art, including, without limitation, oral solutions, injectable formulations, tablets, gel capsules, chewing gum formulations, and more.

Oral solutions and elixir formulations can be simple, or they can be quite complex, involving many types of excipients including water-soluble organic solvents, water-insoluble organic solvents, surfactants, buffers, sugars, flavors, sweeteners, aromatics, dyes, antioxidants, and preservatives. The solutions can include water or be free of water. Organic cosolvents are normally used to solubilize poorly water-soluble drugs, including cannabidiol, to the desired concentration in oral solutions, as is known in the art.

Embodiments of oral solutions include formulations comprising cannabidiol dissolved in a water insoluble solvent, such as long-chain triglycerides, peanut oil, corn oil, soybean oil, sesame oil, olive oil, peppermint oil, hydrogenated vegetable oils, hydrogenated soybean oil, and medium-chain triglycerides. Medium-chain triglycerides can be synthetic or derived from coconut oil and palm seed oil. Other water-insoluble solvents include beeswax, dl-α-tocopherol (Vitamin E), and oleic acid.

When in the form of an oral solution, cannabidiol or a pharmaceutically acceptable prodrug, derivative, salt or solvate thereof can be present in a concentration of from about 50, 75, 100, 150, or 200 mg/mL. Additionally, or alternatively, the oral solution can contain cannabidiol in a concentration of up to about 350, 300, or 250 mg/mL. In some embodiments, the oral solution contains cannabidiol in an amount of from 50 to 125 mg/mL, e.g., about 100 mg/mL.

When in the form of an injectable formulation, cannabidiol or a pharmaceutically acceptable prodrug, derivative, salt or solvate thereof, can be present at a concentration as high as 350, 300, or 250 mg/mL and/or as low as 200, 150 or 100 mg/mL.

In one embodiment, the pharmaceutically active agent is present at a concentration of about 250 mg/mL.

The present compositions contemplate using one or more pharmaceutically active agents chosen from the group comprising cannabidiol and its pharmaceutically acceptable prodrugs, derivatives, salts and solvates.

### Additional Pharmaceutically Active Agents and Excipients

Recitation of cannabidiol and/or its pharmaceutically acceptable prodrugs, derivatives, salts and solvates herein does not preclude combination therapies involving other pharmaceutically active agents. It also does not preclude compositions that comprise additional pharmaceutically active agents. However, in some embodiments, only cannabidiol is used to treat or prevent recurrent pericarditis. In some embodiments, cannabidiol is administered in combination with a therapeutic dose of another active agent, e.g., nonsteroidal anti-inflammatory drugs, aspirin, steroids, colchicine, and/or anakinra. In some embodiments, the cannabidiol is used to treat recurrent pericarditis that is not adequately controlled by other agents.

The present compositions can also contain additional pharmaceutically acceptable excipients, provided that these ingredients do not undermine the basic and novel properties of the invention. For example, oral solutions according to the invention can contain an effective amount of at least one lipophilic antioxidant for improved shelf life. The at least one antioxidant can be selected from the group consisting of vitamin E (also referred to as α-tocopherol), carotenoids (xanthophylls and carotenes), propyl gallate, lecithin, ascorbyl palmitate, butylated hydroxyanisole (BHA), butylated hydroxytoluene (BHT), and monothioglycerol tert-butylhydroquinone (TBHQ). What constitutes an “effective amount” depends on the identity of the compound. In general, the total amount of antioxidant(s) will be more than about 0.01% w/v and less than about 1.5% w/v. In some embodiments, the amount is more than about 0.1% w/v and less than about 1% w/v.

Additional excipients which are contemplated for use in the practice of the disclosure are those available to those of ordinary skill in the art, for example, those found in the United States Pharmacopeia Vol. II and National Formulary Vol. XVII, U.S. Pharmacopeia Convention, Inc., Rockville, Md. (1989).

### Methods of Treatment and Administration

To treat or prevent recurrent pericarditis, cannabidiol and/or its pharmaceutically acceptable prodrugs, derivatives, salts and solvates can be administered in a variety of ways known in the art, including orally and parenterally. The term “parenteral” is meant to include, without limitation, administration via intravenous (IV), subcutaneous (SC), intraperitoneal (IP), and intramuscular (IM) injection. The dose of cannabidiol when administered parenterally can be lower than the dose when administered orally, given that orally administered cannabidiol usually has lower bioavailability.

Parenteral compositions can be administered at least once a month, at least once every two weeks, at least once weekly, or at least once every 6, 5, 4, 3, 2 or 1 day(s). The parenteral composition can also be administered at least twice daily. Oral compositions can be administered at least once a week, at least once every 2 or 1 day(s), or at least twice a day.

For parenteral compositions, at least about 0.01, 0.1, 1, 2, 3, 4, 5, 6, 7, 8, 9, or 10 mg cannabidiol per kg body weight and/or up to about 30, 28, 26, 24, 22, or 20 mg cannabidiol per kg body weight can be administered per dose. Preferably, the dose is at least 1 mg cannabidiol per kg body weight.

For oral compositions, at least about 0.1, 1, 5, 10, 15, 20, 25, or 30 mg cannabidiol per kg body weight can be administered per dose.

The treatment period can be for 6, 5, 4, 3, or 2 months, or for 8, 7, 6, 5, 4, 3, 2, or 1 week(s). In the case of chronic conditions (e.g. recurrent pericarditis), it is envisioned that the treatment period can also be indefinite.

In one embodiment, wherein the pharmaceutically active agent is administered orally, the administration is twice daily at a dose of from about 10 mg/kg. In another embodiment, wherein the pharmaceutically active agent is administered subcutaneously, the administration is at least once a week at a dose of about 5-10 mg/kg body weight per administration.

### EXAMPLES

The following experiments were performed using synthetic cannabidiol (Cas. No. 13956-29-1) having a purity of 100%±2%.

In the following description of the experiments and/or figures, the following abbreviations are used.
CNT=control
LPS=Lipopolysaccharide
ATP=Adenosine Triphosphate
CBD=cannabidiol (synthetically synthesized, pharmaceutical grade)
NaCl=normal saline or saline
Veh=vehicle

### Example 1A—Effect of 10 μM CBD on level of IL-1β in in vitro NLRP3 Inflammasome Activation Model

An experiment was performed to determine the effect of 10 μM cannabidiol on NLRP3 inflammasome activation in an in vitro model as follows.

J774A.1 mouse macrophages were cultured in 96-well plates (15,000 cells/well) and divided into the following groups to which various treatments described below were administered during a period of six hours:
1. Group 1 (CNT)—cells received nothing during the 6-hour treatment period.
2. Group 2 (LPS)—cells received LPS (1 μg/ml) for 6 hours.
3. Group 3 (ATP)—cells received nothing for 5.5 hours and then ATP (5 mM) for the last 30 minutes.
4. Group 4 (LPS+ATP)—cells received LPS (1 μg/mL) for 5.5 hours and ATP (5 mM) for the last 30 minutes.
5. Group 5 (CBD)—cells received cannabidiol solubilized in methanol (10 μM) for 6 hours.
6. Group 6 (CBD+LPS+ATP)—cells received cannabidiol solubilized in methanol (10 μM) and LPS (1 μg/mL) for 5.5 hours and ATP (5 mM) for the last 30 minutes.

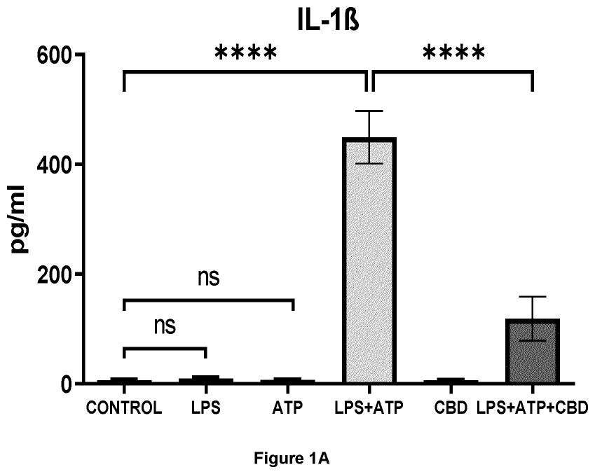

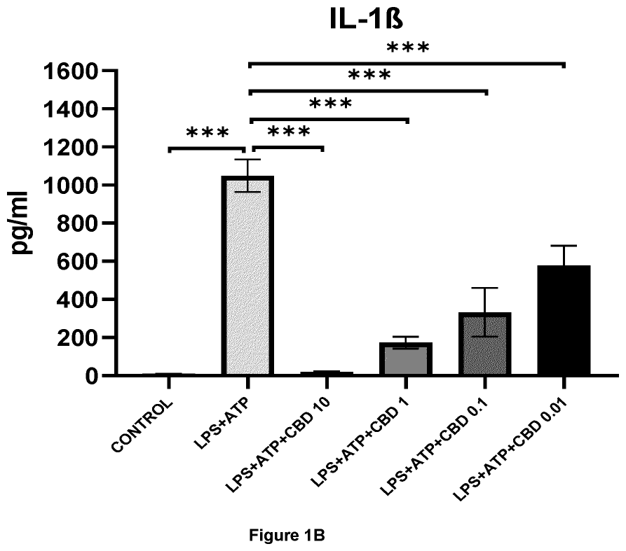

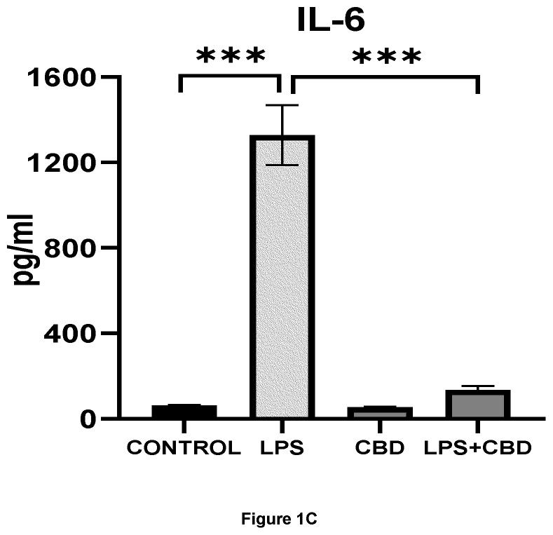

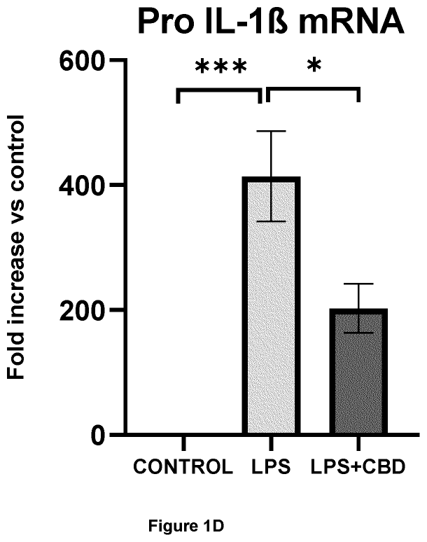

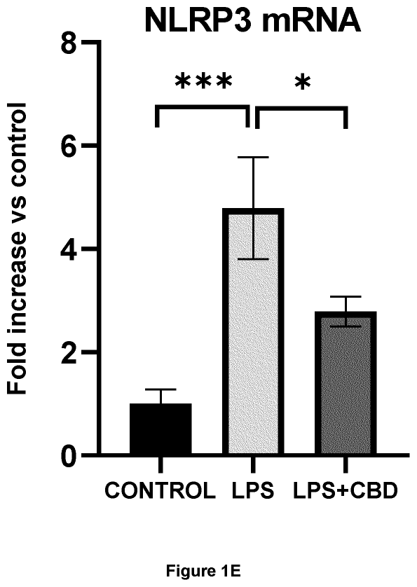

After 6 hours, 100 μL of a sample from each well of cells was collected and loaded as a single point in a specific ELISA assay that was used to measure the amount of IL-1β secreted by the macrophages in each sample. IL-1β is an indicator of and positively correlated with NLRP3 inflammasome activation. The results are summarized in Table 1A.1 and FIG. 1A.

TABLE 1A.1

| | | | | | |
|---|---|---|---|---|---|
| | | | LPS + | | CBD + |
| CNT | LPS | ATP | ATP | CBD | LPS + ATP |

Concentration (pg/ml)

| | | | | | | |
|---|---|---|---|---|---|---|
| | 6.5 | 8.6 | 6.4 | 500.7 | 5.9 | 120.1 |
| | 6.2 | 12.7 | 5.5 | 306.1 | 6.1 | 95 |
| | 6.1 | 8.9 | 6.8 | 479.2 | 5.9 | 226.3 |
| | 6.7 | 9.7 | 7.4 | 510.5 | 6.1 | 33.5 |
| Mean | 6.375 | 9.975 | 6.525 | 449.125 | 6 | 118.725 |
| SEM | 0.13768926 | 0.93752778 | 0.39869579 | 48.1209652 | 0.05773503 | 40.20825734 |

FIG. 1A shows that the cells of Group 4 (LPS+ATP) had significantly higher concentrations of IL-1β compared to the cells of Group 1 (CNT). The cells of Group 6 (CBD+LPS+ATP) had significantly lower concentrations of IL-1β than the cells of Group 4 (LPS+ATP). These results show that cannabidiol was effective to attenuate the increase in IL-1β induced by administration of LPS and ATP in this in vitro model of NLRP3 inflammasome activation of J774A.1 mouse macrophages.

Table 1A.1 shows that cannabidiol attenuated the increase in IL-1β by over 70% calculated as follows:

$$449.13 [ LPS + ATP ] - 6.38 [ CNT ] = 442.75$$

$$118.73 [ LPS + ATP + CBD ] - 6 \text{.38} [ CNT ] = 112 \text{.35}$$

$$[ ( 44 \; 2.75 - 112.35 ) \div 44 \; 2.75 ] \times 100 = 74.62 \%$$

The experiments were repeated, and the results are shown in Tables 1A.2 and 1A.3 below.

TABLE 1A.2

| | | | | | |
|---|---|---|---|---|---|
| | | | | | LPS + |
| | | | LPS + | | ATP + |
| CNT | LPS | ATP | ATP | CBD | CBD |

Concentration (pg/ml)

| | | | | | |
|---|---|---|---|---|---|
| 9.79 | 69.06 | 9.13 | 486.3 | 9.46 | 61.46 |
| 11.31 | 66.78 | 10.8 | 809.26 | 10.46 | 14.53 |
| 9.46 | 35.4 | 9.3 | 569.63 | 8.97 | 13.98 |
| 10.12 | 87.41 | 9.63 | 332.73 | 7.86 | 13.44 |
| 9.13 | 90.48 | 9.13 | 548.07 | 6.35 | 13.44 |
| 8.49 | 58.16 | 8.65 | 467.63 | 8.81 | |

TABLE 1A.3

| | | | | | |
|---|---|---|---|---|---|
| | | | | | LPS + |
| | | | LPS + | | ATP + |
| CNT | LPS | ATP | ATP | CBD | CBD |

Concentration (pg/ml)

| | | | | | |
|---|---|---|---|---|---|
| 7.55 | 44.48 | 8.65 | 224.78 | 6.49 | 17 |
| 7.71 | 50.93 | 7.71 | 480.38 | 8.02 | 11.31 |
| 7.4 | 42.22 | 7.09 | 312.05 | 8.02 | 18.96 |
| 7.71 | 37.07 | 7.09 | 475.47 | 7.4 | 10.8 |
| 7.24 | | 7.4 | 404.9 | 16.04 | |

The mean and SEM values for the data in Tables 1A.2 and 1A.3 are shown in Table 1A.4 below.

TABLE 1A.4

| | | | | | |
|---|---|---|---|---|---|
| | | | LPS + | | LPS + |
| CNT | LPS | ATP | ATP | CBD | ATP + CBD |

Concentration (pg/ml)

| | | | | | | |
|---|---|---|---|---|---|---|
| Mean | 8.71909091 | 58.199 | 8.59818182 | 464.654545 | 8.89818182 | 19.4355556 |
| SEM | 0.4036593 | 6.280082 | 0.35355292 | 46.6490946 | 0.80301004 | 5.04793327 |

Table 1A.4 shows that cannabidiol attenuated the increase in IL-1β by about 97.65%, i.e., over 97%, calculated as follows:

$$464.65 [ LPS + ATP ] - 8.72 [ CNT ] = 455.93$$

$$19.44 [ LPS + ATP + CBD ] - 8.72 [ CNT ] = 10.72$$

$$[ ( 45 \; 5.93 - 10.72 ) \div 45 \; 5.93 ] \times 100 = 97.65 \%$$

### Example 1B—Effect of Different Concentrations of Cannabidiol on Level of IL-1β in In Vitro NLRP3 Inflammasome Activation Model

An experiment was performed to evaluate the effect of different doses of cannabidiol (10, 1, 0.1, and 0.01 μM) using the in vitro model of Example 1A. In this experiment, J774A.1 mouse macrophages were cultured in 96-well plates (15,000 cells/well) and divided into the following groups to which various treatments described below were administered during a period of six hours:
1. Group 1 (Control)—cells received nothing during the 6-hour treatment period.
2. Group 2 (LPS+ATP)—cells received LPS (1 μg/mL) for 5.5 hours and ATP (5 mM) for the last 30 minutes.
3. Group 3 (LPS+ATP+CBD 10)—cells received cannabidiol solubilized in methanol (10 μM) and LPS (1 μg/mL) for 5.5 hours and ATP (5 mM) for the last 30 minutes.
4. Group 4 (LPS+ATP+CBD 1)—cells received cannabidiol solubilized in methanol (1 μM) and LPS (1 μg/mL) for 5.5 hours and ATP (5 mM) for the last 30 minutes.
5. Group 5 (LPS+ATP+CBD 0.1)—cells received cannabidiol solubilized in methanol (0.1 μM) and LPS (1 μg/mL) for 5.5 hours and ATP (5 mM) for the last 30 minutes.
6. Group 6 (LPS+ATP+CBD 0.01)—cells received cannabidiol solubilized in methanol (0.01 μM) and LPS (1 μg/mL) for 5.5 hours and ATP (5 mM) for the last 30 minutes.

After 6 hours, 100 μL of a sample from each well of cells was collected and loaded as a single point in a specific ELISA assay that was used to measure the amount of IL-1β in each sample. The results are summarized in FIG. 1B which shows that cannabidiol was effective to attenuate the increase in IL-1β in a dose dependent fashion, i.e. higher concentrations of cannabidiol led to higher levels of attenuation of IL-1β secretion. In this experiment, groups corresponding to Groups 2, 3, and 5 of Example 1A (LPS alone, ATP alone, and CBD alone) were not tested because they were already tested in Example 1A and found to be no different than the Control group, which was expected since LPS alone, ATP alone, and CBD alone were not expected to activate the NLRP3 inflammasome.

### Example 1C—Effect of 10 μM Cannabidiol on IL-6, pro-IL-1 mRNA, and NLRP3 mRNA in an In Vitro Model of Inflammation

Experiments were performed to evaluate cannabidiol's anti-inflammatory activity and mechanism of action. The secretion of IL-6 and the transcription of the NLRP3 inflammasome components, pro-IL-1β mRNA, and NLRP3 mRNA, are regulated by the NF-κβ pathway and activated by LPS.

In a first experiment, J774A.1 mouse macrophages were cultured in 96-well plates (15,000 cells/well) and divided into the following groups to which various treatments described below were administered during a period of six hours:
1. Group 1 (Control)—cells received nothing during the 6 hours treatment period.
2. Group 2 (LPS)—cells received LPS (1 μg/ml) for 6 hours.
3. Group 3 (CBD)—cells received cannabidiol solubilized in methanol (MetOH) (10 μM) for 6 hours.
4. Group 4 (LPS+CBD)—cells received cannabidiol solubilized in methanol (10 μM) and LPS (1 μg/mL) for 6 hours.

After 6 hours, 100 μL of a sample from each well of cells was collected and loaded as a single point in a specific ELISA assay that was used to measure the amount of IL-6 in each sample. The results are summarized in FIG. 1C which shows that LPS significantly increased the level of IL-6 secreted by the macrophages and that cannabidiol attenuated this increase completely. Without wishing to be bound by theory, it is possible that the anti-inflammatory effect of cannabidiol goes beyond its inhibition of the NLRP3 inflammasome pathway as IL-6 secretion has been shown to be independent of the NLRP3 inflammasome pathway. A second experiment was performed to evaluate the effect of cannabidiol on the transcription of components of the NLRP3 inflammasome, namely, pro-IL-1β mRNA and NLRP3 mRNA. In this second experiment, J774A.1 mouse macrophages were cultured in individual plates (1,000,000 cells/well) and divided into the following groups to which various treatments described below were administered during a period of six hours.
1. Group 1 (Control)—cells received nothing during the 6 hours treatment period.
2. Group 2 (LPS)—cells received LPS (1 μg/ml) for 6 hours.
3. Group 3 (LPS+CBD)—cells received cannabidiol solubilized in methanol (10 μM) and LPS (1 μg/mL) for 6 hours.

At the end of the six-hour treatment period, samples from each group were collected and processed using Polymerase Chain Reaction (PCR) to measure the level of transcription of pro-IL-1B mRNA and NLRP3 mRNA in the samples. The results are summarized in FIGS. 1D and 1E. As shown in these figures, LPS increased the level of transcription of these components of the NLRP3 inflammasome, and cannabidiol (10 μM) significantly attenuated these increases. These results further support cannabidiol's efficacy in reducing inflammation and further illustrate its effect on the NLRP3 inflammasome. Since pericarditis involves (at least) the activation of the NLRP3 inflammasome, suppression of transcription of its components shows that cannabidiol may be useful in treating or preventing pericarditis, including recurrent pericarditis.

### Example 2—Testing Cannabidiol In Vivo

Experiments were conducted using a mouse model of pericarditis described in Mauro et al. (10), which mouse model is incorporated herein by reference for its disclosure of the mouse model.

Adult Institute of Cancer Research (IRC) mice (10 weeks of age) supplied by Harlan Laboratories (Harlan Sprague Dawley Inc.) were used and divided into the following treatment groups, with each group consisting of 4-5 mice:
1. Group 1 (NaCl+Veh)—mice received normal saline (0.9% w/v NaCl) in the surgical procedure described below and were subsequently treated for seven days with a vehicle consisting of ethanol:Cremophor EL:water (1:1:18).
2. Group 2 (NaCl+)—mice received normal saline (0.9% w/v NaCl) in the surgical procedure described below and were subsequently treated for seven days with cannabidiol at a dose of 10 mg/kg. The cannabidiol was dissolved in the same vehicle as the vehicle used for Group 1.
3. Group 3 (Zym+Veh)—mice received Zymosan A in the surgical procedure described below and were subsequently treated for seven days with the same vehicle as the vehicle used for Group 1.
4. Group 4 (Zym+CBD 10 mg/kg)—mice received Zymosan A in the surgical procedure described below and were subsequently treated for seven days with cannabidiol at a dose of 10 mg/kg. The cannabidiol was dissolved in the same vehicle as the vehicle used for Group 1.
5. Group 5 (Zym+CBD 1 mg/kg)—mice received Zymosan A in the surgical procedure described below and were subsequently treated for seven days with cannabidiol at a dose of 1 mg/kg. The cannabidiol was dissolved in the same vehicle as the vehicle used for Group 1.

### Surgical Preparation of the Mice

The mice of all four treatment groups were anesthetized, intubated orotracheally, and placed in the right lateral decubitus position. A left thoracotomy was then performed. Under direct visualization and using a 30 Gauge needle, 1 mg of Zymosan A (referred to herein also as “zymosan”) dissolved in 50 μl of sterile normal saline (0.9% w/v NaCl) was delivered into the pericardial space of the mice of Groups 3 and 4 by carefully lifting the pericardial sac with forceps until a complete distribution of the solution into the pericardium was achieved. Sham procedures were performed vis-a-vis the mice of Groups 1 and 2 by injecting an equal volume of sterile normal saline instead of the Zymosan A solution. All mice received analgesia for the peri-operative period.

For all groups, treatments with either cannabidiol dissolved in vehicle, or vehicle alone, were administered intraperitoneally (IP) every day for seven days with the first treatment administered approximately 30 minutes after the surgery.

### Transthoracic Echocardiographv

On day seven, echocardiography was performed on the mice of all groups under light anesthesia using a Prospect™ T1 ultrasound imaging system (Scintica, London, Canada). In this procedure, the left ventricle in the parasternal short-axis view at the mid-ventricular level was visualized in the bi-dimensional mode (B-mode). The image was optimized for the anterior wall, and the image was zoomed in to visualize the anterior pericardial structures. After optimization of the image, amono-dimensional mode (M-mode) was acquired for optimal spatial-temporal resolution. An investigator blinded to group allocation measured the maximal pericardial space between the 2 layers of the pericardium in both M- and B-mode. The pericardial space is a one-dimensional measure of pericardial effusion.

The results for Groups 1-4 are summarized in Table 2.

TABLE 2

| | | | |
|---|---|---|---|
| Saline + Veh | Saline + CBD | Zym + Veh | Zym + CBD |

Measured pericardial space (mm)

| | | | | |
|---|---|---|---|---|
| | 0.065 | 0.098 | 0.402 | 0.103 |
| | 0.085 | 0.115 | 0.248 | 0.152 |
| | 0.12 | 0.117 | 0.158 | 0.112 |
| | 0.098 | 0.097 | 0.237 | 0.104 |
| | 0.123 | 0.105 | | |
| Mean | 0.0982 | 0.1064 | 0.26125 | 0.11775 |
| SEM | 0.01087842 | 0.00416653 | 0.05101858 | 0.01159292 |

These results show that mice of Group 3 (Zym+Veh) developed a significantly larger pericardial space compared with the mice of Group 1 (Saline+Veh). The mice of Group 2 (Saline+CBD) and of Group 4 (Zym+CBD) did not develop a pericardial space greater than that of the mice of Group 1 (Saline+Veh) as the differences among these groups were not statistically significant.

Cannabidiol attenuated the increase in the measured pericardial space by at least 85%, calculated as follows:

$$0.26125 [ Zym + Veh ] - 0.0982 [ Saline + Veh ] = 0 \text{.16305}$$

$$0.1175 [ Zym + CBD ] - 0.0 \; 982 [ Saline + Veh ] = 0.0 \; 1955$$

$$[ ( 0. 16305 ) - ( 0.01955 ) ] \div [ 0. 163 \; 05 ] \times 100 = about 88.01 \%$$

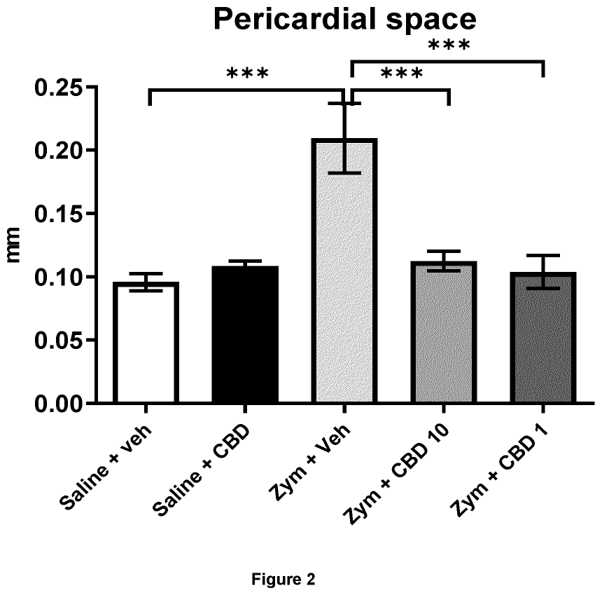

The above experiment was repeated using a lower dose of 1 mg/kg cannabidiol (Group 5 mice). In this later experiment, it was found that cannabidiol, at this lower dose, also attenuated the increase in the measured pericardial space to the same degree as cannabidiol at a dose of 10 mg/kg in the prior experiment. The results of both experiments are shown in FIG. 2 which shows no statistical difference between the pericardial space measured for the control Group 1 (Saline+Veh) and the pericardial space measured for each of Group 4 (Zym+CBD 10 mg/kg) and Group 5 (Zym+CBD 1 mg/kg). Thus, these results show that cannabidiol was effective in attenuating an increase in pericardial space caused by zymosan administration in this mouse model of pericarditis.

### Histochemistry

Following echocardiography, and on day seven, the mice were sacrificed, and their hearts were explanted and processed for pathology. Formalin-fixed paraffin-embedded transverse sections of the hearts were stained with hematoxylin and eosin. An investigator blinded to group allocation measured the pericardial thickness with the aid of computer morphometry using Image-Pro™ Plus 6.0 software (Media Cybernetics, Silver Spring, MD).

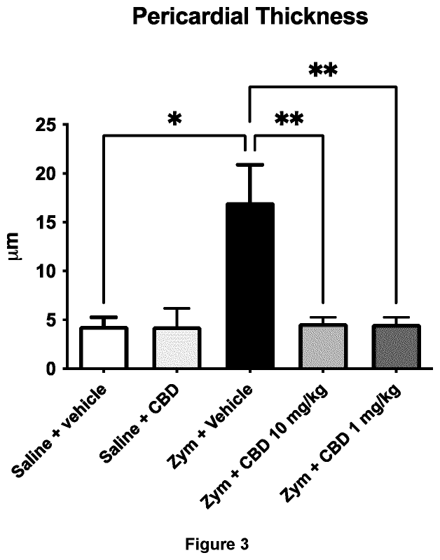

The results are summarized in FIG. 3.

These results show that the mice of Group 3 (Zym+Veh) developed significantly larger pericardial thickness compared with the mice of Group 1 (NaCl+Veh). The mice of Group 2 (NaCl+CBD), Group 4 (Zym+CBD 10 mg/kg)), and Group 5 (Zym+CBD 1 mg/kg) did not develop a pericardial thickness different from that of the mice of Group 1 (NaCl+Veh) as the differences among these groups were not statistically significant. These results show that cannabidiol was effective in both attenuating and preventing an increase in pericardial thickness caused by zymosan administration in this mouse model of pericarditis.

### Human Equivalent Dose

The human equivalent dose of 10 mg/kg and 1 mg/kg used above in mice is 0.81 mg/kg and 0.081 mg/kg, respectively (21). This dose would be applicable to parenteral formulations since cannabidiol was administered parenterally in the above in vivo mouse model.

### Statistical Significance

In the experiments described above, data were expressed as continuous variables as mean and standard error, and one-way ANOVA was used to compare between 3 or more groups at each time point, followed by Sidak test for multiple comparisons. P values less than 0.05 are shown using one asterisk (*). P values less than 0.01 are shown using two asterisks (**). P values less than 0.001 are shown using three asterisks (***). P values less than 0.0001 are shown using four asterisks (****).

### List of Items

The following is a non-exhaustive list of items provided by the invention.

Item 1. Use of cannabidiol, or a pharmaceutically acceptable prodrug, derivative, salt, or solvate thereof, in treating or preventing recurrent pericarditis, or in the preparation of a medicament for the treatment or prevention of recurrent pericarditis.

Item 2. Use of cannabidiol in treating or preventing recurrent pericarditis, or in the preparation of a medicament for the treatment or prevention of recurrent pericarditis.

Item 3. The use of item 1 or 2, wherein the cannabidiol, or pharmaceutically acceptable prodrug, derivative, salt, or solvate thereof, or medicament, is used to prevent recurrent pericarditis.

Item 4. The use of item 1 or 2, wherein the cannabidiol, or pharmaceutically acceptable prodrug, derivative, salt, or solvate thereof, or medicament, is used to treat recurrent pericarditis.

Item 5. The use of any one of items 1 to 4, wherein the cannabidiol, or pharmaceutically acceptable prodrug, derivative, salt, or solvate thereof, or medicament, is effective to attenuate an increase in pericardial effusion in a subject.

Item 6. The use of any one of items 1 to 4, wherein the cannabidiol, or pharmaceutically acceptable prodrug, derivative, salt, or solvate thereof, or medicament, is effective to prevent an increase in pericardial effusion in a subject.

Item 7. The use of any one of items 1 to 6, wherein the cannabidiol, or pharmaceutically acceptable prodrug, derivative, salt, or solvate thereof, or medicament, is effective to attenuate an increase in pericardial thickness in a subject.

Item 8. The use of any one of items to 1 to 6, wherein the cannabidiol, or pharmaceutically acceptable prodrug, derivative, salt, or solvate thereof, or medicament, is effective to prevent an increase in pericardial thickness in a subject.

Item 9. The use of any one of items 1 to 8, wherein the cannabidiol, or pharmaceutically acceptable prodrug, derivative, salt, or solvate thereof, or medicament, is effective to attenuate or prevent an increase in the level of at least one of interleukin-1β (IL-1β), interleukin 6 (IL-6), transcription of pro-IL-1β mRNA, and transcription of NLRP3 mRNA, in a subject.

Item 10. The use of item 9, wherein the level of at least one of interleukin-1β (IL-1) and interleukin 6 (IL-6) is attenuated by at least about 50, 55, 60, 65, 70, 75, 80, 85, 90, or 95% as compared to the level that would be present if the cannabidiol, or pharmaceutically acceptable prodrug, derivative, salt, or solvate thereof, or medicament, was not administered to the subject.

Item 11. The use of any one of items 1 to 10, wherein the cannabidiol or pharmaceutically acceptable prodrug, derivative, salt, or solvate thereof, or medicament, is effective to attenuate an increase in pericardial effusion by at least about 50, 55, 60, 65, 70, 75, 80, 85, 90, or 95%, or up to about 100% as compared to the amount of pericardial effusion that would be present if the cannabidiol, or pharmaceutically acceptable prodrug, derivative, salt, or solvate thereof, or medicament, was not administered to the subject.

Item 12. The use of any one of items 1 to 11, wherein the cannabidiol or pharmaceutically acceptable prodrug, derivative, salt, or solvate thereof, or medicament, is effective to attenuate an increase in pericardial thickness by at least about 50, 55, 60, 65, 70, 75, 80, 85, 90, or 95%, or up to about 100% as compared to the pericardial thickness that would be present if the cannabidiol, or pharmaceutically acceptable prodrug, derivative, salt, or solvate thereof, or medicament, was not administered to the subject.

Item 13. The use of any one of items 1 to 12, wherein the cannabidiol, or pharmaceutically acceptable prodrug, derivative, salt, or solvate thereof, is synthetic.

Item 14. A method of treating or preventing recurrent pericarditis in a subject in need thereof, comprising administering an effective amount of cannabidiol, or a pharmaceutically acceptable prodrug, derivative, salt, or solvate thereof, to the subject.

Item 15. A method of treating or preventing recurrent pericarditis in a subject in need thereof, comprising administering an effective amount of cannabidiol to the subject.

Item 16. The method of item 14 or 15, wherein the cannabidiol, or pharmaceutically acceptable prodrug, derivative, salt, or solvate thereof is administered orally.

Item 17. The method of item 16, wherein the cannabidiol, or pharmaceutically acceptable prodrug, derivative, salt, or solvate thereof is administered at least once a week, at least once every 2 or 1 day(s), or at least twice a day.

Item 18. The method of item 16 or 17, wherein at least about 0.1, 1, 5, 10, 15, 20, 25, or 30 mg cannabidiol per kg body weight is administered per dose.

Item 19. The method of any one of items 16 to 18, wherein cannabidiol is administered twice daily at a dose of from about 10 mg/kg body weight.

Item 20. The method of item 14 or 15, wherein the cannabidiol, or pharmaceutically acceptable prodrug, derivative, salt, or solvate thereof is administered parenterally.

Item 21. The method of item 20, wherein the cannabidiol, or pharmaceutically acceptable prodrug, derivative, salt, or solvate thereof is administered at least once a month, at least once every two weeks, at least once weekly, or at least once every 6, 5, 4, 3, 2 or 1 day(s).

Item 22. The method of item 21, wherein the cannabidiol, or pharmaceutically acceptable prodrug, derivative, salt, or solvate thereof is administered at least twice daily.

Item 23. The method of any one of items 20 to 22, wherein at least about 0.01, 0.1, 1, 2, 3, 4, 5, 6, 7, 8, 9, or 10 mg cannabidiol per kg body weight and/or up to about 30, 28, 26, 24, 22, or 20 mg cannabidiol per kg body weight is administered per dose.

Item 24. The method of item 23, wherein the dose is at least 1 mg cannabidiol per kg body weight.

Item 25. The method of item 20, wherein cannabidiol is administered subcutaneously at least once a week at a dose of about 5-10 mg/kg body weight.

Item 26. The method of any one of items 14 to 25, wherein the cannabidiol, or pharmaceutically acceptable prodrug, derivative, salt, or solvate thereof, is effective to prevent recurrent pericarditis.

Item 27. The method of any one of items 14 to 25, wherein the cannabidiol, or pharmaceutically acceptable prodrug, derivative, salt, or solvate thereof, is effective to treat recurrent pericarditis.

Item 28. The method of any one of items 14 to 27, wherein the cannabidiol, or pharmaceutically acceptable prodrug, derivative, salt, or solvate thereof, is effective to attenuate an increase in pericardial effusion in the subject.

Item 29. The method of any one of items 14 to 27, wherein the cannabidiol, or pharmaceutically acceptable prodrug, derivative, salt, or solvate thereof, is effective to prevent an increase in pericardial effusion in the subject.

Item 30. The method of any one of items 14 to 29, wherein the cannabidiol, or pharmaceutically acceptable prodrug, derivative, salt, or solvate thereof, is effective to attenuate an increase in pericardial thickness in the subject.

Item 31. The method of any one of items 14 to 29, wherein the cannabidiol, or pharmaceutically acceptable prodrug, derivative, salt, or solvate thereof, is effective to prevent an increase in pericardial thickness in the subject.

Item 32. The method of any one of items 14 to 31, wherein the cannabidiol, or pharmaceutically acceptable prodrug, derivative, salt, or solvate thereof, is effective to attenuate or prevent an increase in the level of at least one of interleukin-1β (IL-1), interleukin 6 (IL-6), transcription of pro-IL-1β mRNA, and transcription of NLRP3 mRNA, in the subject.

Item 33. The method of item 32, wherein the level of at least one of interleukin-1β (IL-β) and interleukin 6 (IL-6) is attenuated by at least about 50, 55, 60, 65, 70, 75, 80, 85, 90, or 95% as compared to the level that would be present if the cannabidiol, or pharmaceutically acceptable prodrug, derivative, salt, or solvate thereof was not administered to the subject.

Item 34. The method of any one of items 14 to 33, wherein the cannabidiol, or pharmaceutically acceptable prodrug, derivative, salt, or solvate thereof, is effective to attenuate an increase in pericardial effusion by at least about 50, 55, 60, 65, 70, 75, 80, 85, 90, or 95%, or up to about 100% as compared to the amount of pericardial effusion that would be present if the cannabidiol, or pharmaceutically acceptable prodrug, derivative, salt, or solvate thereof was not administered to the subject.

Item 35. The method of any one of items 14 to 34, wherein the cannabidiol, or pharmaceutically acceptable prodrug, derivative, salt, or solvate thereof, is effective to attenuate an increase in pericardial thickness by at least about 50, 55, 60, 65, 70, 75, 80, 85, 90, or 95%, or up to about 100% as compared to the amount of pericardial effusion that would be present if the cannabidiol, or pharmaceutically acceptable prodrug, derivative, salt, or solvate thereof was not administered to the subject.

Item 36. The method of any one of items 14 to 35, further comprising the step of identifying a subject suffering from or at risk of suffering from recurrent pericarditis, prior to said administration.

Item 37. The method of any one of items 14 to 36, wherein the cannabidiol, or pharmaceutically acceptable prodrug, derivative, salt, or solvate thereof, is synthetic.

Item 38. Cannabidiol, or a pharmaceutically acceptable prodrug, derivative, salt, or solvate thereof, for use in the treatment or prevention of recurrent pericarditis.

Item 39. Cannabidiol for use in the treatment or prevention of recurrent pericarditis.

Item 40. The cannabidiol, or pharmaceutically acceptable prodrug, derivative, salt, or solvate thereof, of item 38 or 39, for use in preventing recurrent pericarditis.

Item 41. The cannabidiol, or pharmaceutically acceptable prodrug, derivative, salt, or solvate thereof, of item 38 or 39, for use in treating recurrent pericarditis.

Item 42. The cannabidiol, or pharmaceutically acceptable prodrug, derivative, salt, or solvate thereof, of any one of items 38 to 41, for use in attenuating an increase in pericardial effusion in a subject.

Item 43. The cannabidiol, or pharmaceutically acceptable prodrug, derivative, salt, or solvate thereof, of any one of items 38 to 41, for use in preventing an increase in pericardial effusion in a subject.

Item 44. The cannabidiol, or pharmaceutically acceptable prodrug, derivative, salt, or solvate thereof, of any one of items 38 to 43, for use in attenuating an increase in pericardial thickness in a subject.

Item 45. The cannabidiol, or pharmaceutically acceptable prodrug, derivative, salt, or solvate thereof, of any one of items 38 to 43, for use in preventing an increase in pericardial thickness in a subject.

Item 46. The cannabidiol, or pharmaceutically acceptable prodrug, derivative, salt, or solvate thereof, of any one of items 38 to 45, for use in attenuating or preventing an increase in the level of at least one of interleukin-1β (IL-1β), interleukin 6 (IL-6), transcription of pro-IL-1β mRNA, and transcription of NLRP3 mRNA in a subject.

Item 47. The cannabidiol, or pharmaceutically acceptable prodrug, derivative, salt, or solvate thereof, of any one of items 38 to 46, wherein the cannabidiol, or pharmaceutically acceptable prodrug, derivative, salt, or solvate thereof, is synthetic.

Item 48. A composition comprising an effective amount of cannabidiol, or a pharmaceutically acceptable prodrug, derivative, salt, or solvate thereof, and an effective amount of a delivery vehicle, wherein the composition is for use in treating or preventing recurrent pericarditis in a subject in need thereof.

Item 49. A composition comprising an effective amount of cannabidiol, and an effective amount of a delivery vehicle, wherein the composition is for use in treating or preventing recurrent pericarditis in a subject in need thereof.

Item 50. The composition of item 48 or 49, wherein the composition is for use in treating recurrent pericarditis in a subject in need thereof.

Item 51. The composition of item 48 or 49, wherein the composition is for use in preventing recurrent pericarditis in a subject in need thereof.

Item 52. The composition of any one of items 48 to 51, wherein the composition is an oral solution, and wherein the cannabidiol or a pharmaceutically acceptable prodrug, derivative, salt or solvate thereof is present in a concentration of from about 50, 75, 100, 150, or 200 mg/mL and/or up to about 350, 300, or 250 mg/mL.

Item 53. The composition of item 52, wherein the oral solution contains cannabidiol in an amount of from 50 to 125 mg/mL.

Item 54. The composition of item 53, wherein the oral solution contains cannabidiol in an amount of about 100 mg/mL.

Item 55. The composition of any one of items 48 to 51, wherein the composition in an injectable formulation, and wherein the cannabidiol or a pharmaceutically acceptable prodrug, derivative, salt or solvate thereof is present at a concentration up to about 350, 300, or 250 mg/mL and/or from about 200, 150 or 100 mg/mL.

Item 56. The composition of item 55, wherein the cannabidiol or a pharmaceutically acceptable prodrug, derivative, salt or solvate thereof is present at a concentration of about 250 mg/mL.

The above experiments show that cannabidiol may represent a novel strategy for treating and preventing recurrent pericarditis. The embodiments described above are by way of example only and are not intended to limit the scope of the invention as described herein and defined by the following claims.

### REFERENCES

1. Adler Y, Charron P, Imazio M, et al., 2015 ESC Guidelines for the diagnosis and management of pericardial diseases: the Task Force for the Diagnosis and Management of Pericardial Diseases of the European Society of Cardiology (ESC) Endorsed by: The European Association for Cardio-Thoracic Surgery (EACTS). Eur Heart J 2015; 36:2921-64.
2. Imazio M, Gaita F, LeWinter M., Evaluation and treatment of pericarditis: a systematic review. JAMA 2015; 314:1498-506.
3. Bonaventura A, Montecucco F., Inflammation and pericarditis: are neutrophils actors behind the scenes?J Cell Physiol 2019; 234:5390-8.
4. Buckley L F, Viscusi M M, Van Tassell B W, Abbate A., Interleukin-1 blockade for the treatment of pericarditis. Eur Heart J Cardiovasc Pharmacother 2018; 4:46-53.
5. Toldo S, Abbate A., The NLRP3 inflammasome in acute myocardial infarction. Nat Rev Cardiol 2018; 15:203-14.
6. Mauro A G, Bonaventura A, Mezzaroma E, Quader M, Toldo S., NLRP3 Inflammasome in Acute Myocardial Infarction. J Cardiovasc Pharmacol 2019; 74:175-87.
7. Chiabrando J G, Bonaventura A, Vecchie A, et al., Management of acute and recurrent pericarditis: JACC State-of-the-Art Review. J Am Coll Cardiol 2020; 75:76-92.
8. Bayes-Genis A, Adler Y, de Luna A B, Imazio M., Colchicine in pericarditis. Eur Heart J 2017; 38: 1706-9.
9. Brucato A, Imazio M, Gattorno M, et al., Effect of anakinra on recurrent pericarditis among patients with colchicine resistance and corticosteroid dependence: the AIRTRIP randomized clinical trial. JAMA 2016; 316: 1906-12.
10. Mauro et al., The Role of NLRP3 Inflammasome in Pericarditis. JACC: BASIC TO TRANSLATIONAL SCIENCE Vol. 6, No. 2, 2021, pp. 137-150; doi.org/10.1016/j.jacbts.2020.11.016
11. “FDA Approves First Treatment for Disease That Causes Recurrent Inflammation in Sac Surrounding Heart”. U.S. Food and Drug Administration (FDA). 18 Mar. 2021 (fda.gov/drugs/news-events-human-drugs/fda-approves-first-treatment-disease-causes-recurrent-inflammation-sac-surrounding-heart).
12. Klein et al. Clinical Burden and Unmet Need in Recurrent Pericarditis: A Systematic Literature Review. Cardiology in Review; Vol. 30, No. 2, March/April 2022.
13. Liu et al. Inhibitory Effect of Cannabidiol on the Activation of NLRP3 Inflammasome Is Associated with Its Modulation of the P2X7 Receptor in Human Monocytes. J. Nat. Prod. 2020, 83, 6, 2025-2029 Publication Date: May 6, 2020. doi.org/10.1021/acs.jnatprod.0c00138.
14. Libro et al., (2016) Cannabidiol Modulates the Immunophenotype and Inhibits the Activation of the Inflammasome in Human Gingival Mesenchymal Stem Cells. Front.

Physiol.7:559. doi: 10.3389/fphys.2016.00559.
15. Suryavanshi, S. V.; Zaiachuk, M.; Pryimak, N.; Kovalchuk, I.; Kovalchuk, O., Cannabinoids Alleviate the LPS-Induced Cytokine Storm via Attenuating NLRP3 Inflammasome Signaling and TYK2-Mediated STAT3 Signaling Pathways In Vitro. Cells 2022, 11, 1391. doi.org/10.3390/cells11091391.
16. Suryavanshi S V, Kovalchuk I and Kovalchuk O, (2021) Cannabinoids as Key Regulators of Inflammasome Signaling: A Current Perspective. Front. Immunol. 11: 613613. doi: 10.3389/fimmu.2020.613613.
17. Stanley Christopher P., Hind William H., O'Sullivan Saoirse E., Is the cardiovascular system a therapeutic target for cannabidiol?Br J Clin Pharmacol/75: 2/313-322. doi: 10.1111/j.1365-2125.2012.04351.x
18. Atalay Sinemyiz, Jarocka-Karpowicz Iwona, and Skrzydlewska Elzbieta, Antioxidative and Anti-Inflammatory Properties of Cannabidiol. Antioxidants 2020, 9, 21; doi:10.3390/antiox9010021.
19. Sunda Falone, Arowolo Afolake, A molecular basis for the anti-inflammatory and anti-fibrosis properties of cannabidiol. The FASEB Journal. 2020; 00:1-10. doi: 10.1096/fj.202000975R.
20. Garza-Cervantes J. A., Ramos-Gonzilez M, Lozano O, Jerjes-Sinchez C, Garcia-Rivas G., Therapeutic Applications of Cannabinoids in Cardiomyopathy and Heart Failure.

Hindawi Oxidative Medicine and Cellular Longevity Volume 2020, Article ID 4587024, 17 pages. doi.org/10.1155/2020/4587024.
21. Nair Anroop B., Jacob Shery, A simple practice guide for dose conversion between animals and human. J Basic Clin Pharm. March 2016-May 2016; 7(2): 27-31. doi: 10.4103/0976-0105.177703

1.-13. (canceled)

14. A method of treating or preventing recurrent pericarditis in a subject in need thereof, comprising administering an effective amount of cannabidiol, or a pharmaceutically acceptable prodrug, derivative, salt, or solvate thereof, to the subject.

15. (canceled)

16. The method of claim 14, wherein said administration is effective to prevent recurrent pericarditis.

17. The method of claim 14, wherein said administration is effective to treat recurrent pericarditis.

18. The method of claim 14, wherein said administration is effective to attenuate or prevent an increase in pericardial effusion in the subject.

19. (canceled)

20. The method of claim 1, wherein said administration is effective to attenuate or prevent an increase in pericardial thickness in the subject.

21. (canceled)

22. The method of claim 14, wherein said administration is effective to attenuate an increase in the level of at least one of interleukin-1β (IL-1β), interleukin 6 (IL-6), transcription of pro-IL-1β mRNA, and transcription of NLRP3 mRNA, in the subject.

23. The method of claim 22, wherein said administration causes the level of at least one of interleukin-1β (IL-1β) and interleukin 6 (IL-6) to be attenuated by at least about 50, 55, 60, 65, 70, 75, 80, 85, 90, or 95%.

24. The method of claim 14, wherein said administration is effective to attenuate an increase in pericardial effusion by at least about 50, 55, 60, 65, 70, 75, 80, 85, 90, or 95%, or up to about 100%.

25. The method of claim 14, wherein said administration is effective to attenuate an increase in pericardial thickness by at least about 50, 55, 60, 65, 70, 75, 80, 85, 90, or 95%, or up to about 100% as compared to the pericardial thickness that would be present if the cannabidiol, or pharmaceutically acceptable prodrug, derivative, salt, or solvate thereof was not administered to the subject.

26. (canceled)

27. The method of claim 14, wherein the cannabidiol, or pharmaceutically acceptable prodrug, derivative, salt, or solvate thereof, is synthetic.

28.-41. (canceled)

42. The method of claim 14, wherein the cannabidiol, or pharmaceutically acceptable prodrug, derivative, salt, or solvate thereof is administered orally.

43. The method of claim 42, wherein the cannabidiol, or pharmaceutically acceptable prodrug, derivative, salt, or solvate thereof is administered at least once a week, at least once every 2 or 1 day(s), or at least twice a day.

44. The method of claim 42, wherein at least about 1, 5, 10, 15, 20, 25, or 30 mg cannabidiol per kg body weight is administered per dose.

45. The method of claim 42, wherein cannabidiol is administered twice daily at a dose of from about 10 mg/kg body weight.

46. A composition comprising an effective amount of cannabidiol, or a pharmaceutically acceptable prodrug, derivative, salt, or solvate thereof, and an effective amount of a delivery vehicle, wherein the composition is for use in treating or preventing recurrent pericarditis in a subject in need thereof.

47. The composition of claim 46, wherein the composition is an oral solution, and wherein the cannabidiol or a pharmaceutically acceptable prodrug, derivative, salt or solvate thereof is present in a concentration of from about 50, 75, 100, 150, or 200 mg/mL and/or up to about 350, 300, or 250 mg/mL.

48. The composition of claim 47, wherein the oral solution contains cannabidiol in an amount of from 50 to 125 mg/mL.

49. The composition of claim 48, wherein the oral solution contains cannabidiol in an amount of about 100 mg/mL.
